(19) United States
(12) Patent Application Publication
(10) Pub. No.: US 2002/0086061 A1
(43) Pub. Date: Jul. 4, 2002

(54) Preparation of micron-size felodipine particles by microfluidization

(76) Inventors: Vinay K. Sharma, Long Valley, NJ (US); Arun K. Srivastav, Maharashtra (IN); Javed Hussain, Maharashtra (IN); Habil F. Khorakiwala, Mumbai (IN)

Correspondence Address: SCHWEGMAN, LUNDBERG, WOESSNER & KLUTH, P.A., P.O. Box 2938, Minneapolis, MN, 55402, US

(21) Appl. No.: 09/765,726

(22) Filed: Jan. 18, 2001

Related U.S. Application Data
(63) Continuation-in-part of application No. 09/697,670, filed on Oct. 26, 2000.
(60) Provisional application No. 60/240,923, filed on Jan. 18, 2000.
(60) Provisional application No. 60/240,924, filed on Oct. 26, 1999.

(51) Int. Cl. A61K 31/724; A61K 9/14; A61K 9/50

(52) U.S. Cl. 424/499; 514/58

(57) ABSTRACT

The present invention provides the components for a stable felodipine composition and a process for preparing the composition. The composition includes felodipine, non-covalently bound to β-cylodextrin, and an optional binder as a moisture carrier component for the migration of hydroxide ions to the non-covalently bound felodipine and β-cylodextrin. The felodipine composition is combined with a carrier comprising cyclodextrin particles, a water-insoluble alkaline component and a swellable polymer.

### CROSS-REFERENCE TO RELATED APPLICATIONS

This application is a continuation-in-part of U.S. patent application Ser. No. 09/697,670, filed Oct. 26, 2000, which claims priority from U.S. provisional patent application Serial No. 60/______,______, which was filed on Oct. 26, 1999 as U.S. patent application Ser. No. 09/427,231, for which a petition under 37 C.F.R §1.53(c) to convert the non-provisional application to a provisional application was filed on Aug. 29, 2000, and U.S. provisional patent application Ser. No. 60/______,______, which was filed on filed Jan. 18, 2000 as U.S. patent application Ser. No. 09/484,573, for which a petition under 37 C.F.R §1.53(c) to convert the non-provisional application to a provisional application was filed on Aug. 29, 2000, all of which are incorporated herein by reference.

### BACKGROUND OF THE INVENTION

A commercially available oral dosage form of felodipine, ethyl methyl (RS)-4-(2,3-dichloropentyl)-1,4-dihydro-2,6-dimethypyridine-3,5-dicarboxylate, is Plendil® ER Tablets. This product is believed to be prepared according to the disclosure in U.S. Pat. No. 4,803,081. The drug is dissolved or dispersed in an effective amount of a semi-solid or liquid nonionic solubilizer (active compound and the solubilizer are in a preferred ratio range from 1:2 to 1:6). A preferred solubilizer is polyethoxylated castor oil (e.g., Cremophor® RH 40 by BASF). Unfortunately, Cremophor® was implicated in embryo toxicity and allergic reactions. Sharma, A. et. al., Int. J. Cancer 71, 103-107, (1997).

It was reported by Schnadelbach, D. Chairman, European Pharmacopoeia, Published by the European Department for the Quality of Medicines within the Council of Europe, Strassbourg, 3rd Edition, 1997, pp. 847-848, and in British Pharmacopoeia 1998, Volume I, pages 574-575, (1998), that an impurity described as impurity “A” is one of the three impurities that can be found in felodipine during analysis using liquid chromatography. The chemical structure of felodipine and this degradation product, impurity A, are shown in the formula below:

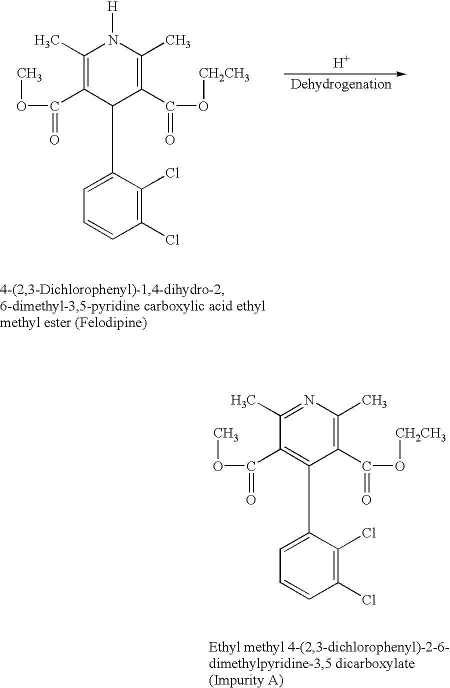

It was hypothesized that felodipine undergoes acid-catalyzed, solvolytic oxidation in a solid state due to the degradation of dicalcium phosphate dihydrate to form dehydrogenated felodipine (Impurity A). Other impurities (Impurity B and Impurity C) were not significantly increased during the testing of the current formulation placed at accelerated conditions. Impurity B is the dimethyl ester of felodipine, dimethyl 4-(2,3-dichloropentyl)-1,4-dihydro-2,6-dimethypyridine-3,5-dicarboxylate, and Impurity C is the diethyl ester of felodipine, diethyl 4-(2,3-dichloropentyl)-1,4-dihydro-2,6-dimethypyridine-3,5-dicarboxylate.

### SUMMARY OF THE INVENTION

The present invention provides a method for preparing a unit dosage form of a stable felodipine composition. The method comprises forming a drug containing core by compressing a composition comprising granulated microparticles, of felodipine and cyclodextrin, having a diameter of from about 0.5 microns to about 9 microns, and a carrier comprising cyclodextrin particles, a water-insoluble alkaline component and a swellable polymer. The unit dosage form can be optionally coated with a resilient membrane coating. The composition includes felodipine, non-covalently bound to β-cyclodextrin, a source of hydroxide ions, and an optional binder as a moisture carrier component for the migration of hydroxide ions to the non-covalently bound felodipine and β-cylodextrin.

Inorganic excipients such as dicalcium phosphate (DCP) dihydrate can undergo hydrolysis and thereby serve as a source of hydrogen ions which can cause solid-state, solvolytic oxidation of felodipine into “Impurity A”. The present invention discloses that β-cyclodextrin can be used as a primary stabilizing component in a formulation containing felodipine. Therefore, dicalcium phosphate dihydrate is eliminated and replaced by β-cyclodextrin.

Magnesium trisilicate is added to provide a source of hydroxide ions. It is believed that the hydroxyethyl cellulose hydrates and absorbs hydroxide ions which then can migrate to the weakly acidic β-cyclodextrin. Felodipine particles can be non-covalently bound to β-cylodextrin, for example, during microfluidization. The felodipine particle is protected from acid-catalyzed oxidation (including abnormal acidity from water or air) because in the β-cylodextrin micro-environment, the pH is intentionally designed to be alkaline in nature.

The aqueous process of granulation is environmentally compatible, in contrast with solvent-based granulation processes disclosed in the prior art. The use of cyclodextrins, and especially β-cylodextrin to stabilize pharmaceutical compounds and compositions, especially during microfluidization of Felodipine, has not been reported in the literature.

### BRIEF DESCRIPTION OF THE FIGURES

FIG. 1 is a graphic illustration of a comparison of heat treatment and screening of suspensions of various excipients, combinations of excipients and combinations of felodipine and excipients to determine the source of hydrogen ions.

FIGS. 2 and 2A schematically illustrate the process for the preparation of the Felodipine compositions of the invention.

FIG. 3 is a graphic illustration of the comparative data of two discreetly different, drug particle diameters.

FIG. 4 is a graphic illustration of the release profile of the formulation 9C, tested in the Bioequivalence Study 1.

FIG. 5 is a graphic illustration of the effect of concentration of magnesium trisilicate on drug release by locating the magnesium trisilicate in the “bowl charge” of the Fluid Bed Granulator Dryer.

FIG. 6 is a graphic illustration of the influence of locating unmicronized and powder magnesium trisilicate in the “bowl charge” vs. Spraying it as a non-microfluidized slurry along with drug dispersion, on RSD of drug release.

FIG. 7 is a graphic illustration of the influence of the ratio of β-cylodextrin to felodipine in microfluidized dispersion on drug release for 5 mg dosage using the tablets prepared in examples 6 and 7.

FIG. 8 is a graphic illustration of the bioequivalence of felodipine tablet of the invention compared to a commercial felodipine tablet, Plendil.

### DETAILED DESCRIPTION OF THE INVENTION

The present invention is motivated by the undesirability of Cremophor® in pharmaceutical compositions where it can be eliminated. The invention involves a novel use of the process known as “Microfluidization” for achieving bioequivalence to Plendil®. The process is described in copending U.S. patent application Ser. No. 09/340,917, filed Jun. 28, 1999, titled “Preparation of Micron-Size Pharmaceutical Particles by Microfluidization.” The application describes a process where micronized feed materials are microfluidized at low pressures (e.g., about 3,500 to 7,000 or 4,000 to 6,000 pounds per square inch) to effectively prepare particles in the 6-12 micron size range, using from 1-3 passes through the microfluidizer.

The compositions of the present invention are substantially free of dicalcium phosphate. As used herein, “substantially free” means less than about 1%, and typically less than 0.6%, of the composition by weight is dicalcium phosphate.

The present invention provides a drug delivery system for drugs having low water solubility, such as, for example, felodipine. The invention uses a monophasic particle size distribution, ranging from 1-3 microns, to provide a swellable, erosion rate-controlled drug delivery system. This system uses a combination of a highly swellable non-ionic polymer and hydrophilic insoluble excipients.

In another embodiment the present invention provides a process for preparing a kinetically stable, controlled release formulation of felodipine by preparing dispersions of hydrophilized alkaline material and the hydrophobic drug non-covalently bonded to a cyclodextrin. The sequential processing may comprise the granulating of a blend of a macroparticulate cyclodextrin and a medium viscosity (1,000-6,500 cps), highly swellable hydroxyalkyl cellulose using dispersions of alkaline material and hydrophobic drug (felodipine). A compressed core is formed by conventional core pressing of a mixture comprising granulated microparticles of felodipine and cyclodextrin, having a diameter of from about 0.5 microns to about 9 microns and an optional binder; wherein the felodipine particles are non-covalently bonded to the cyclodextrin; and a carrier comprising cyclodextrin particles, a water-insoluble alkaline component, and a (medium viscosity) swellable polymer.

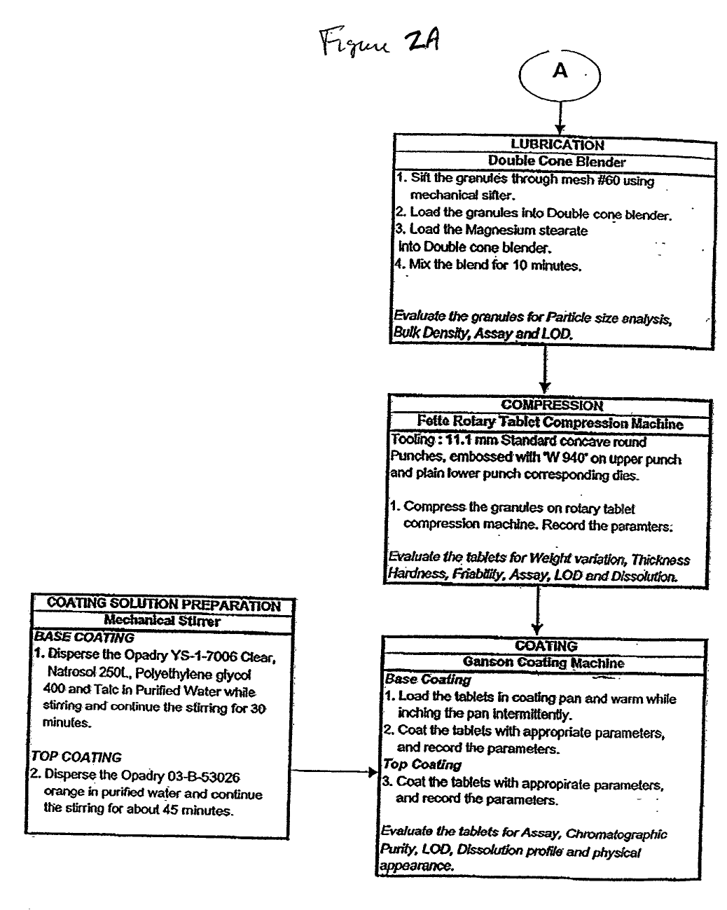

An illustrative process for preparing representative tablets of the invention is described in FIGS. 2 and 2A. The tablets of the invention are compressed granules prepared from a microfluidized latex formed from felodipine, a binder, such as hydroxypropyl cellulose (HPC), a dissolution enhancer, such as β-cyclodextrin (β-CD) and a polydimethylsiloxane-silicon dioxide defoamer, such as simethicone in water. The latex is granulated in a fluidized bed with an alkaline agent, such as magnesium trisilicate, additional β-CD, HPC, hydroxyethyl cellulose (HEC) (swellable polymer for release control) and simethicone, to provide granules having a matrix that erodes uniformly. An example of the compositions of the granulate is shown on Table 1. The granules are tableted with magnesium stearate. The resulting tablets are coated with two TiO₂-hydroxypropyl methyl cellulose (HPMC) coatings.

TABLE 1
Granulate Composition (Dry)

| Ingredient | Function | Wt-% |
|---|---|---|
| Felodipine | Active | 2.3 |
| β-CD | Solubilization Aid | 71.2 |
| HPC | Binder | 5.8 |
| Simethicone (30%) | Antifoam | 0.01 |
| Mg Trisilicate | alkaline agent | 4.7 |
| HEC | Gelling Agent | 16 |

The geometry of the drug delivery system (e.g., the tablet), at a constant polymer (binder):excipient:drug ratio, can be modified from a generally spherical matrix (e.g., diameter of 10.6 mm and thickness of 6.46 mm, approximately 288 mm²) to provide a more cylindrical form (e.g., diameter of 12.92 mm and a thickness of 4.58 mm, approximately 342 mm²) to generate a larger surface area and a shorter distance for erosion or diffusion of the delivery system. The resultant effect of this particular modification is an acceleration of matrix erosion. The success of any drug delivery system is governed by the drug absorption performance, which is in turn at least a partial function of the drug release rate and characteristics. This is particularly true where the drug permeability after oral administration is not a rate-limiting step in the process of the distribution of drug in the body.

In one example of the microfluidization process, an aqueous medium is used for wet-micronization of the drug/excipient mixture, based on the particle size distribution (PSD) of the feed material (unmicronized vs. micronized) and the targeted particle size distribution of the outflow micro-suspension. A granulated drug containing material is subsequently obtained by preparing two phases. The first phase is a microfluidized mixture of the drug (latex) a cyclodextrin, such as, β-cylodextrin and a water soluble binder, in water. The microfluidized phase (latex) is subsequently blended with a separately prepared dispersion of hydroxypropyl cellulose in water, and an optional antifoaming agent, such as simethicone.

Microfluidization facilitates the reduction of the mean particle size of the drug and β-cylodextrin mixture and creates a smooth latex-like micro-suspension. In the presence of cyclodextrins, resulting particle sizes are in the nanometer range, preferably less than 400 nm, more preferably less than 500 nm, and most preferably less than 1000 nm.

The preferred range of particle sizes is from about 500 to about 4000 nm. More preferable are particle sizes from about 1000 to about 3000 nm. Most preferable are particle sizes from about 1000 to about 1500 nm. Mixtures within these ranges can be produced according to the process described herein (with or without the presence of surface-active agents or surface modifying-agents). The cyclodextrins, as noted herein, remain as particles within the microfluidized system and the resultant product. The cyclodextrin particles may or may not be separable from the pharmaceutical particles.

The literature, for example, Jozsef Szejtli, Pharmaceutical Technology, June 1991, reports that cyclodextrins (CDS) are enzymatically modified starches made up of glucopyranose units. Three different CDS are known. CDS feature a cylinder-shaped, macro-ring structure with a large internal axial cavity. They are crystalline and non-hygroscopic. The outer surface of a CD molecule is hydrophilic, but the internal cavity is apolar. When a molecule of another substance, e.g., a drug such as felodipine, is placed into this cavity an inclusion complex is formed. No covalent bonding is involved. This is in contrast to the β-cylodextrin that is on the surface of the pharmaceutical particles.

The dissolution rate of a solid is described by the Noyes-Whitney equation:

$$\frac{ⅆ c}{ⅆ t} = k ( {C}_{s} - C )$$

$$Where$$

$$k = \frac{D A}{V h}$$

Where

D=diffusion coefficient

A=Surface area of the dissolving solid

V=Volume of the dissolving solid

h=Diffusion layer thickness

C_{S}=Solute concentration in the diffusion layer

C=Solute concentration in the bulk

During the early phase of dissolution, C_{S}>>C and is essentially equal to the saturation solubility C_{S}. Under these conditions and at constant temperature and agitation, the above equation reduces to:

$$\frac{ⅆ c}{ⅆ t} = k {C}_{s} , \\ Where \\ k = \frac{D A}{V h}$$

The dissolution rate expressed in the above equation is termed the intrinsic dissolution rate and is characteristic of each solid compound in a given solvent under fixed hydrodynamic conditions. The intrinsic dissolution rate in a fixed volume of solvent is generally expressed as mg dissolved in min⁻¹ cm⁻².

An inherent problem of drugs that have low water solubility is low and often insufficient bioavailability. This lack of bioavailability is related to the low saturation solubility C_{S}, and dissolution rate dc/dt. Attempts to solubilize drugs using micelles or cyclodextrins have achieved only limited success. Microfluidization (wet-micronization) of a hydrophobic drug, such as felodipine, in the presence of optimum carriers, such as cyclodextrins, presents a better approach. The primary drug particle size approaches about 1-3 microns.

The sizes described herein for particles, whether for the pharmaceuticals or for the cyclodextrins, are non-aggregated particle sizes. Unless otherwise stated, the sizes are weight average particle sizes. The ranges may alternatively be applied to number average particle sizes, usually where fewer than 10% by number of the particles exceed the stated average size by more than 25%.

The aqueous solubility of felodipine was determined to be about 0.0001% at all pH levels, respectively. The intrinsic dissolution rate of felodipine was calculated to be 0.00086 mg min⁻¹ cm-⁻². The media used was a 500 ml phosphate buffer, Type II, and dissolution was assisted by using a paddle. Based on this data, a 10-20 micron range for felodipine will exhibit dissolution rate-limited absorption.

The use of antifoaming agents, such as silicone compounds, fluorinated compounds, such as simethicone and FC-40 manufactured by Minnesota Mining and Manufacturing Co. (although there are many chemical classes of materials known in the art for this purpose) has already been briefly referred to. These compounds provide a benefit to processing performance. The benefit is unrelated to any surface-active effect between the drug and the liquid carrier used in the microfluidization process. When particles are provided in the aqueous carrier, significant amounts of air or other gas can be carried with the particles. Because of the small size of the particles, the air or other gas is not easily shed from the surface of the small particles. Thus, it can be carried into the carrier liquid, and foaming can occur in the suspension. This is not desirable in the microfluidization process and can adversely affect the ability to control the particle size and other benefits. Therefore it is desirable, either before any microfluidization occurs or shortly after initiation of the microfluidization process, to introduce an anti-foaming agent to the particles and/or to the particles and liquid (water) carrier. It is particularly desirable to add the particles and antifoaming agent to the liquid carrier and allow a significant dwell time (e.g., at least 5 minutes, preferably at least 10 or 15 minutes, up to an hour or more) to allow the air or other gas to disassociate itself from the surface of the particles. Some mild agitation to ‘shake-off’ the bubbles from the surface of the particles may be desirable, but is not essential.

Defoaming may occur directly in the storage or feed tank used in the microfluidization system or may be carried out at another time, prior to introduction of the suspension into the microfluidizer. The defoaming agents, some of which are surfactants (a term that is actually quite broad in scope), are preferably used in amounts that are much smaller than the concentrations or volumes that are usually necessary for effective surface-active properties. For example, defoaming agents may be used in weight/weight percentages of the solution in a range of from about 0.005 to about 0.08% by weight of the total solution/dispersion. Preferably, the defoaming agents are used in a range of from about 0.005 to about 0. 1%. Most preferably the defoaming agents are used in a range of from about 0.0005% to about 0.2%. Conventional surface-active agents are typically used in higher concentrations.

The cyclodextrin is introduced into the carrier drug particle system as a solid particle. The cyclodextrin remains as solid particles in the process, even if there is some breakdown or minor dissolution of the cyclodextrin. Thus, the cyclodextrin does not act as a surface-modifying agent, i.e., it does not form a coating on the surface of the pharmaceutical particle, and remains only associated with the hydrophobic, water-insoluble drug particle, in a non-covalent mixture, during and after the microfluidization process. The pharmaceutical hydrophobic, relatively water-insoluble drug, the cyclodextrin or both may be added to the suspension or used to form the suspension in any size particles, such as, for example, from about 1 to about 50, preferably from about 1 to about 100, and most preferably from about 1 to about 200 micrometers in size. The cyclodextrin particles may be larger or smaller than the drug particles. The cyclodextrin (preferably β-cylodextrin) may be added to the drug in a ratio of drug to cyclodextrin of from about 1:50 to about 50:1. A preferred ratio of drug to cyclodextrin is from about 1:50 to about 20:1. A more preferred ratio of drug to cyclodextrin is from about 1:30 to about 5:1. Even more preferred is a ratio of drug to cyclodextrin from about 1:25 to about 1:1. The most preferred ratio of drug to cyclodextrin is from about 1:15 to about 1:2.

A stable felodipine composition may be based on micronized and microfluidized felodipine, a cyclodextrin, preferably β-cylodextrin, a binder (e.g., hydroxypropyl cellulose, preferably Klucel® LF) and a matrix-forming material or gelling agent (e.g., hydroxyethyl cellulose, preferably (Natrosol® 250M)), and optionally preferably an alkaline excipient, preferably magnesium trisilicate, and an optional lubricant, preferably magnesium stearate (stearic acid is preferably specifically avoided).

The granules are prepared by placing β-cylodextrin and hydroxyethyl cellulose in a FBGD (Fluid Bed Granulator Dryer) insert of GPCG-5 ( a chemical processing unit [Model 5] sold by Glatt Air Techniques, Mahwah, N.J.). The microfluidized drug dispersion containing the binder, (e.g., hydroxypropyl cellulose), and magnesium trisilicate is sprayed onto the blended material in the GPCG-5 FBGD (Fluid Bed Granulator Dryer) container. The granules are dried to a moisture content of not more than about 2%. The granules are lubricated with colloidal silicon dioxide, and magnesium stearate. The product is compressed into single dosage forms (tablets) using 11 mm standard concave punches.

The compressed core can be film coated with a combination of low viscosity (5-125 cps) hydroxyalkyl cellulose polymers, appropriately plasticized to form a resilient membrane that will not rupture due to the various stresses that may be created within the film structure and the core matrix. The core matrix is intentionally designed to include a swellable polymer, which has viscoelastic tableting behavior. By using this type of polymer, the matrix develops an elastic recovery-related stress after ejection from the tablet machine. Hence, a resilient film is useful to minimize film rupture. A suitable core coating is a cosmetic membrane composed of low viscosity hydroxyethyl cellulose (Natrosol® 250 L), described in U.S. patent application Ser. No. 09/579,559 filed May 26, 2000.

The amount of drug, such as, for example, felodipine in each dosage form is preferably from about 0.5 mg to about 25.0 mg. More preferred are dosage forms containing from about 1 mg to about 15 mg of drug. Most preferred are dosage forms containing from about 2.5 mg to about 10 mg of drug.

The cyclodextrins useful in the present invention include but are not limited to α-cyclodextrin, β-cylodextrin, δ-cyclodextrin, dimethyl-β-cyclodextrin and hydroxypropyl-β-cylodextrin. The preferred cyclodextrin is β-cyclodextrin. The amount of cyclodextrin in the composition is from about 50 to about 80 weight percent of the composition based on the total weight of the composition based on the weight of the composition. Preferably the amount of cyclodextrin is from about 60% to about 75%. The most preferred amount of cyclodextrin is from about 60 to about 70 weight percent of the composition based on the total weight of the composition.

The matrix forming material is water soluble (or water-dispersible) and swellable. A preferred matrix-forming material comprises a hydroxy alkyl cellulose which is commercially available as various grades such as Natrosol® 250 M (_{MW} 720,000, MPA 4500-6500 for a 2% aqueous dispersion), and Natrosol® 250 H (_{MW} 1,000,000, MPA 1500-2500 for a 1% aqueous dispersion). The preferred grade is Natrosol® 250M. The concentration of matrix-forming material in the granules may range from 10-40% of the weight of the granules. Preferred proportions are 12-18% and most preferred is a range of 15-16%.

The basic alkaline agents useful in the present invention can be selected from the group consisting of oxides, or hydroxide, carbonate, and trisilicate salts of strong basic cations such as Mg²⁺, Ca²⁺, A1³⁺ and the like. These are preferably pH 9 or greater. Non-limiting examples of suitable alkaline materials include materials such as, magnesium oxide, magnesium trisilicate, aluminum hydroxide, magnesium hydroxide, magnesium aluminum silicate (Veegum®) and the like. The preferred basic alkaline material is magnesium trisilicate.

The concentration of the alkaline agent in the granule may range from about 0.5 to about 15% of the weight of the composition (granule). Preferably the concentration of the alkaline agent in the composition is from about 2 to about 10%. More preferably the concentration of the alkaline agent in the composition is from about 3 to about 8%. Most preferably the concentration of the alkaline agent in the composition is about 5%.

To study the cause solid-state-degradation based on a dry blend stability experimental design, stability of felodipine was studied at 60° C. and 40° C./75% relative humidity (RH).

The process of the present invention enhances the effective dissolution rate of drugs such as felodipine which has an M×(Log₁₀)×P computed value of 3.22, a MW (molecular weight) of 384.26, and an extremely low aqueous solubility of 0.5 mg/ml.

In the present invention, a unique micronization approach has been taken to provide non-agglomerated materials to increase the efficiency of drug absorption. Microfluidizer processors rely upon the forces of shear, impact and agitation to deagglomerate and disperse a solid into a liquid. The process takes place at relatively high energy conditions within an interaction chamber (IXC) and may employ an additional chamber called an Auxiliary Processing Module (APM).

The core of the delivery system comprises microparticulate felodipine with a particle diameter of 0.5 to 10.0 microns, in a matrix comprised of highly swellable hydroxyethyl cellulose, a cyclodextrin such as, for example, β-cyclodextrin, and hydrophilized magnesium trisilicate. A microfluidized dispersion can have a specific surface area of 5.5 m²/g or greater. Felodipine may be complexed with cyclodextrin, β-cyclodextrin, dimethyl-β-cyclodextrin, and hydroxypropyl β-cylodextrin to enhance solubility and stability. The preferred cyclodextrin is β-cylodextrin and is present as 30-80 percent w/w of the active core of a delivery system. The general range for felodipine is about 0.5-3% w/w whereas the preferred range is 0.6-2.1% w/w. The general range for the highly swellable matrix binder (e.g., the hydroxyethyl cellulose) is about 17 to 69.5% w/w.

A pharmaceutically acceptable binder is used to prepare a mass of suitable consistency, which after drying will retain its structure until compressed. Pharmaceutically acceptable binders include natural and synthetic adhesives, by way of non-limiting examples including materials such as sodium alginate, soluble cellulosic materials such as sodium carboxymethyl cellulose, methyl cellulose, and hydroxypropyl cellulose, and polyvinyl pyrrolidone. All dissolve in water to give clear, viscous preparations. The preferred binder is hydroxypropyl cellulose and the preferred range is 3-6% w/w.

The composition of this invention can contain a swellable polymer, which is hydrophilic in nature. The polymer is based on hydroxypropylmethyl cellulose or hydroxyethyl cellulose or other gelling agents such as alginates, carrageenan, pectin, guar gum, xanthan gum, modified starch, sodium carboxymethyl cellulose and hydroxypropyl cellulose. This list is not meant to be exclusive. The preferred swellable polymer is hydroxyethyl cellulose. The preferred grade is Natrosol 250M (traded by Aqualon, Wilmington, Del.). The preferred range is 10-30% w/w.

It is desirable to use magnesium trisilicate in its hydrophilized form. This is achieved either by preparing a slurry of about 100 mesh fine magnesium trisilicate powder in a 2% dispersion of hydroxypropyl cellulose (HPC) or microfluidizing it to a finer particle size such as 10-20 microns resembling a viscous suspension in feel and consistency.

A hydrophilizing agent for magnesium trisilicate may be a low viscosity polymer such as hydroxypropyl methyl cellulose, hydroxypropyl cellulose or hydroxyethyl cellulose. The role of the hydrophilizing agent in case of magnesium trisilicate is to decrease the anti-bonding influence of magnesium trisilicate on the compaction of the matrix. When added in the form of dry powder, especially as a larger, free flowing particle to the “bowl charge” of the fluid bed granulator dryer (FBGD), magnesium trisilicate acts as an anti-bonding lubricant. Magnesium trisilicate may be used in a concentration of 1-15 percent based on the weight of the tablet. The preferred concentration is 3-5% w/w. The magnesium trisilicate is located as a fine powder or as a slurry and then sprayed into the bowl. The preferred way of locating it is in the slurry or even better as a microfluidized, viscous suspension. A further preferred approach is to locate magnesium trisilicate in the slurry prepared by microfluidization in the presence of β-cylodextrin (1:1 to 1:3).

Magnesium trisilicate should not be included in the granulating medium because it counterbalances the binding influence of the granulating medium by behaving as a lubricant. For example, when it was included at a concentration 3-5% of the tablet weight in the granulating medium, the tablet structure became softer regardless of the moisture content and its particle size.

These cores are designed to be highly swellable and erodible in the presence of gastrointestinal fluids. Traditional attempts to create a matrix with high compressibility and high gel strength will defeat the purpose of adequately delivering an extremely hydrophobic drug. Atmospheric stress conditions will require protection of the matrix achieved by hydrophilization of magnesium trisilicate, separation of drug-loaded granulating medium from magnesium trisilicate slurry, low moisture in the granulation prior to compression and finally the elastic covering which serves as a containment package for the internally stressed formulation.

The act of compression presses the granules against the die wall and punch faces will such a force so that the core can be difficult to eject and can have a rough surface if the lubricant is not included. External lubricants such as stearates of divalent metals like magnesium, calcium and zinc function by coating the surface of the granules with a film, which reduces interfacial friction between the granules and the compressing surfaces. The preferred lubricant for this invention is magnesium stearate and the preferred range is 0.25-0.75% w/w of the tablet or core.

The use of antifoaming agents, such as silicone compounds can provide a benefit to the process performance that is unrelated to any surface active effect they may have on the relationship of the pharmaceutical to the liquid carrier in the microfluidization process. When particles are provided in the aqueous carrier, significant amounts of air or other gas is carried with the particles. Because of the small size of the particles, it is carried into the carrier liquid, and the foaming can occur in suspension. This is highly undesirable in the microfluidization process and adversely affects the ability of the process to control the particle size and other benefits. Therefore it is desirable, either before any microfluidization occurs or shortly after initiation of the microfluidization process, to introduce an anti-foaming agent to the particles and/or to the particles and liquid (water) carrier. It is particularly desirable to add the particles and antifoaming agent to the liquid carrier and allow a significant dwell time (e.g., at least 5 minutes, preferably at least 10 or 15 minutes, up to an hour or more) to allow the air or other gas to disassociate itself from the surface of the particles. Some mild agitation to “shake-off” the bubble from the surface of the particles may be desirable, but is not essential. This defoaming may occur directly within storage or feed tank for use in the microfluidization system or may be done at another time prior to introduction of the suspension into the microfluidizer. The defoaming agents, some of which are surfactants, may also be used, and are preferably used in amounts that are much smaller than the concentrations or volumes that are usually necessary for effective surface active properties. For example, defoaming agents may be used in wt/wt percentages of the dispersion in ranges, for example, of about 0.010-0.030 by weight of the total dispersion. Preferably the defoaming agent is less than about 0.030 wt %.

When the specific formulation under investigation was coated with a 7.5% w/w aqueous dispersion of hydroxyethyl cellulose (100-125 mPa's, traded as Natrosol® 250 L), plasticized with 10% w/w of the dry polymer weight polyethylene glycol 400, and Opadry clear, the film stretched with the expansion of the formulation and assumed the shape of the matrix.

### EXAMPLES

The compositions and methods of the present invention will be more fully apparent from consideration of the following specific, non-limiting examples of preferred embodiments of the invention.

### General Procedure for Preparation Felodipine Compositions

1. Hydroxypropyl cellulose, NF, EP, JP (Klucel LF), 242.9 g, was slowly added to a stainless steel (SS) container equipped with stirrer and 3,920 g of purified water while stirring vigorously, to obtain clear mucilage.

2. Felodipine BP micronized (175 g), β-cylodextrin (β-CD) micronized (1487.5 g) and simethicone emulsion (3.5 g) were added to the Klucel LF dispersion from Step 1.

3. The dispersion from Step 2 was passed through a microfluidizer (M-210 EH) at 10,000 psi, single pass.

4. A second Klucel dispersion was prepared by adding Klucel LF, 105 g, to purified water 1187.5 g, while stirring vigorously, to obtain clear mucilage. The drug dispersion from Step 3 is added to this Klucel dispersion.

5. A third Klucel dispersion was prepared by adding Klucel LF (87.5 g) to 2,333 g of purified water while stirring vigorously, to obtain clear mucilage. Magnesium trisilicate, 350 g (100 mesh), 350 g of micronized β-cylodextrin, and simethicone (3.5 g) were added to the third Klucel dispersion and mixed.

6. The drug dispersion from Step 4 and magnesium trisilicate dispersion from Step 5 were transferred into two separate measuring cylinders. The cylinders were connected, sequentially, to a Glatt (GPCG-5) through a peristaltic pump.

7. Charge 3,500 g of unmicronized β-cyclodextrin and 1,100 g of hydroxyethyl cellulose (Natrosol® 250 M) into GPCG-5 granulator container.

8. Using an appropriate air volume, inlet temperature, and spray rate, the material from Step 7 was granulated with the magnesium trisilicate dispersion from Step 5, and followed by the drug dispersion from Step 4.

9. When drug dispersion is complete, the granules were dried to a moisture content of less than 2.5 percent. Stop drying and discharge the product.

10. The magnesium stearate is added to the granules from Step 9 and blend using an appropriate blender.

11. The lubricated granules, from Step 10, are compressed into tablets using a rotary or depression machine equipped with 11 mm standard concave tooling.

12. The tablets are finished by initially applying a resilient base coat comprising hydroxyethyl cellulose and hydroxypropyl methyl cellulose followed by a outer coat.

### Drug Dispersion Preparation

Felodipine was micronized using a (Alpine Jet Mill, Model Hosokawa, Alpine AG, Type K20 M-S60 DR). It was then added, with mixing, to a 3.87% w/w dispersion of hydroxypropyl cellulose in water (Klucel® LF, traded by Alkaline, Division of Hercules, Del.) along with micronized β-cylodextrin (24% w/w of the dispersion), using a Lightning Mixer with a propeller stirrer at medium speed. The particle size distribution, computed using Malvern Mastersizer®, was from about 5-7 microns. The mixture is microfluidized at 10,000 PSI (Microfluidizer™ M-210 EH) in order to achieve a target particle diameter of 2.5-4.0 microns (90% of the particle size is below this range and 50% of the particles are below 1.5-2 microns). Simethicone, 0.05% w/w of the dispersion, was added to prepare a foamless dispersion.

### Magnesium Trisilicate Dispersion Preparation

Magnesium trisilicate hydrate (100 mesh) was dispersed in a dispersion of about 10% w/w, hydroxypropyl cellulose and about 45% w/w, unmicronized β-cyclodextrin (100 mesh) and Simethicone emulsion (0.05% w/w). The dispersion was continuously stirred using a Lightning Mixer with a propeller stirrer at medium speed. The purpose of treating magnesium trisilicate with β-cyclodextrin was to hydrophilize it in order to create a uniformly wettable dispersion in the fluid bed granulation drying (FBGD) bowl (“bowl charge”).

### Fluid Bed Granulation Drying Operation

The fluid bed granulation drying (FBGD) was conducted using a GPCG-5 processing unit (Model 5) traded by Glatt Air Techniques, Mahwah, N.J. The FBGD bowl is one of the inserts which permits spray granulation operation and drying.

The “bowl charge” is composed of micronized β-cyclodextrin (Cavitron™ 8900, 25 μ) and hydroxyethyl cellulose (Natrosol® 250 M) in about a 3:1 ratio. In a preferred embodiment, the magnesium trisilicate dispersion was sprayed into the “bowl” in order to prime the “bowl charge” as well as microdisperse magnesium trisilicate in the materials of the “bowl charge”. The processing conditions employed are listed in Table 2, Table 3 and Table 4 as follows:

TABLE 2
Processing Conditions:

| | | | |
|---|---|---|---|
| Nozzle Type | Flush | Filter Cleaning | Every 60 Sec. |
| Nozzle Position | 2 | Bag Shaking | 5 Sec./25 Sec. |
| Nozzle Port Size | 1.2 mm | Mode of Shaking | Asynchronous |
| Filter size | 20 Microns | Atomizing Air | 2 Bars (˜432 |
| | | For Spraying | mm) |

TABLE 3
Processing Conditions for Spraying of
Magnesium Trisilicate Dispersion

| | | | Processing | |
|---|---|---|---|---|
| Elapsed | Inlet Air | Product | Air Flow, M³ | Spray Rate, |
| Time, min | Temp, ° C. | Temp, ° C. | /hr | g/min |
| 0-5 | 55 | 36 | 80 | 60 |
| 5-30 | 75 | 25 | 150 | 80 |
| 30-50 | 85 | 38 | 200 | — |

Before loading the material in the “bowl charge”, the plenum was pre-heated to 70° C.

TABLE 4
Processing Conditions for Spraying of Drug Dispersion

| | | | Processing | Spray | |
|---|---|---|---|---|---|
| Elapsed | Inlet Air | Product | Air Flow, | Rate, | |
| Time, min | Temp, ° C. | Temp, ° C. | m³/hr | g/min | LOD, % |
| 0-15 | 55 | 24-27 | 100-150 | 60 | |
| 15-70 | 70 | 25-27 | 200-300 | 80 | |
| 70-95 | 85 | 60 | 350 | — | 2.3 |

Before loading the material in the “bowl charge”, the plenum was pre-heated to 70 degree C.

### Particle Size Distribution Characterization of Granulation

Exemplary results of the size distribution of the particles prepared as described herein are reported in Table 5. The granular particles were sieved using 30 mesh, 40 mesh and 60 mesh sieves. The granular density was determined and illustrated in Table 5A. The Hauser ratio was used to determine compaction. It was determined that 60 mesh particles were the optimum size for compaction.

TABLE 5
Sieve Analysis Data

| Sieve Analysis | Sifting-30 Mesh | Sifting-40 Mesh | Sifting-60 Mesh |
|---|---|---|---|
| +30 | 0 | 0 | 0 |
| −30/+40 | 11.9 | 0 | 0 |
| −40/+60 | 54 | 51.2 | 0.9 |
| −60/+100 | 20.8 | 28.6 | 63.5 |
| −100/+120 | 2.5 | 4.4 | 8.3 |
| −120/+200 | 6.9 | 9.9 | 17.2 |
| −200 | 3.9 | 5.9 | 9.9 |

TABLE 5A
Density

| Granule density, | | | |
|---|---|---|---|
| g/cc | | | |
| Bulk | 0.352 | 0.364 | 0.396 |
| Tap | 0.419 | 0.433 | 0.495 |
| Hausner Ratio | 1.19 | 1.19 | 1.25* |

*For optimum compaction, the Hausner ratio (Tap density/Bulk Density) should be greater than 1.2.

After passing through a 60 mesh screen, the granules were lubricated with 0.5% magnesium stearate (HyQual®), based on the weight of the unlubricated granules. The granules were compressed according to the conditions provided in Table 6.

TABLE 6
Lubrication/Compression:

| | Speed, | | | | |
|---|---|---|---|---|---|
| Fill | Tablets | Precom- | Post- | Precom- | Main |
| Depth, | per | pression | Compression | pression | Compression |
| mm | Hour | Height (mm) | Height (mm) | Force | Force |
| 9 | 50,000 | 2.5 | 1.60 | — | 22 kN |
| 9.2 | 50,000 | 2.5 | 1.60 | 5 kN | 21 kN |

The tablets were coated using a suitable R&D coating pan such as LDCS 3.75L (Vector Corporation, N.J.). The machine configuration included standard nozzle type, a0.2 mm nozzle port, and standard air pattern. Pan rotation was 20 RPM. For batch size ranging from 2-3 kg, the following processing parameters were maintained, after pre-warming is completed. Tables 7 describes the coating compositions and Table 8 describes the processing conditions.

TABLE 7
Film Coating Composition

| | | |
|---|---|---|
| Hydroxyethyl cellulose | 8.60 | 2.00 |
| (Elastic Membrane; | | |
| Natrosol 250 L) | | |
| Talc (Alpha Fill 500) | 0.86 | 0.20 |
| Polyethylene glycol 400 | 0.86 | 0.20 |
| COLOR COAT | 10.75 | 2.50 |
| (Opadry 03-B-53026 | | |
| Orange) | | |

TABLE 8
Processing Conditions

| | | | Processing | | |
|---|---|---|---|---|---|
| | | | Air Flow | Spray | |
| Elapsed | Inlet Air | Product | m³/hr | Rate, | Spray Air, |
| Time, min | Temp, ° C. | Temp, ° C. | (CFM) | g/min | psi |
| 0-30 | 62 | 37 | (41) | 7 | 19 |
| 30-60 | 64 | 41 | (51) | 8 | — |
| Drying | 67 | 44 | (52) | — | — |

### Tablet Dissolution

The dissolution profiles referred to herein were conducted using the method as described in the US Pharmacopeia, Volume XXII, utilizing a Type 2 paddle assembly at 50 rpm. The media used was a pH 6.5 phosphate buffer containing 1% sodium lauryl sulfate (wt/wt). (As used herein, the term “FERT” refers to Felodipine Extended Release Tablets.)

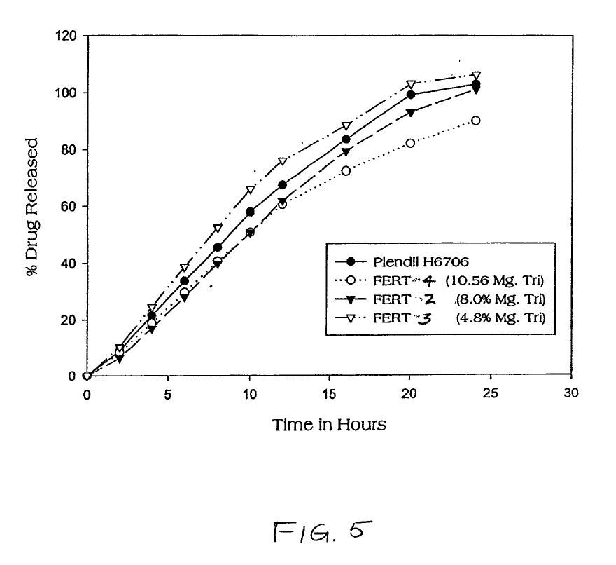

Following the general procedure described herein the several felodipine formulations were prepared and tableted. The amount of each ingredient used is disclosed in Examples 1 to 8. The results of dissolution profiles of formulations FERT 2, FERT 3 and FERT 4, prepared as prepared in Examples 2, 3, and 4, respectively, are illustrated in FIG. 5.

### EXAMPLE 1

| No. | Ingredient | mg/Unit | |
|---|---|---|---|
| 1 | Felodipine, BP, Micronized | 10 | |
| 2 | Hydroxypropyl Cellulose (Klucel ® LF) | 14 | |
| 3 | β-cyclodextrin (Cavitron), Micronized | 85 | |
| 4 | β-cyclodextrin (Cavitron), Unmicronized | 226 | |
| 5 | Magnesium trisilicate | 62 | |
| 6 | Hydroxyethyl Cellulose (Natrosol ® 250M) | 69 | |
| 7 | Simethicone Anti-Foaming Emulsion | 0.12 | |
| | Sub-Total, Unlubricated | 466.12 | |
| 8 | Magnesium Stearate (0.5% of Unlubricated) | 2.3306 | |
| | Sub-Total, Core Weight | | 468.451 |
| 9 | Hydroxyethyl Cellulose (Natrosol ® 250L) | 5.62141 | |
| 10 | Hydroxypropyl methyl Cellulose (Opadry ® | 4.68451 | |
| | Clear) | | |
| 11 | Talc (Alphafil ®) | 0.70268 | |
| 12 | Polyethylene glycol 400 | 0.70268 | |
| | Sub-Coat Weight Gain | | 11.7113 |
| 13 | Opadry ® 03-B-53026 Orange Weight Gain | | 11.7113 |
| | Total, Coated Tablet Weight | | 491.873 |

### EXAMPLE 2

| No. | Ingredient | mg/Unit | |
|---|---|---|---|
| 1 | Felodipine, BP, Micronized | 10 | |
| 2 | Hydroxypropyl Cellulose (Klucel ® LF) | 14 | |
| 3 | β-cyclodextrin (Cavitron), Micronized | 85 | |
| 4 | β-cyclodextrin (Cavitron), Unmicronized | 226 | |
| 5 | Magnesium trisilicate | 35 | |
| 6 | Hydroxyethyl Cellulose (Natrosol ® 250M) | 69 | |
| 7 | Simethicone Emulsion | 0.12 | |
| | Sub-Total, Unlubricated | 439.12 | |
| 8 | Magnesium Stearate (0.5% of Unlubricated) | 2.1956 | |
| | Sub-Total, Core Weight | | 441.316 |
| 9 | Hydroxyethyl Cellulose (Natrosol ® 250L) | 5.29579 | |
| 10 | Hydroxypropyl methyl Cellulose (Opadry ® | 4.41316 | |
| | Clear) | | |
| 11 | Talc (Alphafil ®) | 0.66197 | |
| 12 | Polyethylene glycol 400 | 0.66197 | |
| | Sub-Coat Weight Gain | | 11.0329 |
| 13 | Opadry ® 03-B-53026 Orange Weight Gain | | 11.0329 |
| | Total, Coated Tablet Weight | | 463.381 |

### Example 3

| No. | Ingredient | mg/Unit | |
|---|---|---|---|
| 1 | Felodipine, BP, Micronized | 10 | |
| 2 | Hydroxypropyl Cellulose (Klucel ® LF) | 14 | |
| 3 | β-cyclodextrin (Cavitron), Micronized | 85 | |
| 4 | β-cyclodextrin (Cavitron), Unmicronized | 226 | |
| 5 | Magnesium trisilicate | 20 | |
| 6 | Hydroxyethyl Cellulose (Natrosol ® 250M) | 69 | |
| 7 | Simethicone Emulsion | 0.12 | |
| | Sub-Total, Unlubricated | 424.12 | |
| 8 | Magnesium Stearate (0.5% of Unlubricated) | 2.1206 | |
| | Sub-Total, Core Weight | | 426.241 |
| 9 | Hydroxyethyl Cellulose (Natrosol ® 250L) | 5.11489 | |
| 10 | Hydroxypropyl methyl Cellulose (Opadry ® | 4.26241 | |
| | Clear) | | |
| 11 | Talc (Alphafil ®) | 0.63936 | |
| 12 | Polyethylene glycol 400 | 0.63936 | |
| | Sub-Coat Weight Gain | | 10.656 |
| 13 | Opadry ® 03-B-53026 Orange Weight Gain | | 10.656 |
| | Total, Coated Tablet Weight | | 447.553 |

### Example 4

| No. | Ingredient | mg/Unit | |
|---|---|---|---|
| 1 | Felodipine, BP, Micronized | 10 | |
| 2 | Hydroxypropyl Cellulose (Klucel ® LF) | 14 | |
| 3 | β-cyclodextrin (Cavitron), Micronized | 85 | |
| 4 | β-cyclodextrin (Cavitron), Unmicronized | 226 | |
| 5 | Magnesium trisilicate | 47.5 | |
| 6 | Hydroxyethyl Cellulose (Natrosol ® 250M) | 69 | |
| 7 | Simethicone Emulsion | 0.12 | |
| | Sub-Total, Unlubricated | 451.62 | |
| 8 | Magnesium Stearate (0.5% of Unlubricated) | 2.2581 | |
| | Sub-Total, Core Weight | | 453.878 |
| 9 | Hydroxyethyl Cellulose (Natrosol ® 250L) | 5.44654 | |
| 10 | Hydroxypropyl methyl Cellulose (Opadry ® | 4.53878 | |
| | Clear) | | |
| 11 | Talc (Alphafil ®) | 0.68082 | |
| 12 | Polyethylene glycol 400 | 0.68082 | |
| | Sub-Coat Weight Gain | | 11.347 |
| 13 | Opadry ® 03-B-53026 Orange Weight Gain | | 11.347 |
| | Total, Coated Tablet Weight | | 476.572 |

### Example 5

| No. | Ingredient | mg/Unit | |
|---|---|---|---|
| 1 | Felodipine, BP, Micronized | 10 | |
| 2 | Hydroxypropyl Cellulose (Klucel ® LF) | 24.88 | |
| 3 | β-cyclodextrin (Cavitron), Micronized | 85 | |
| 4 | β-cyclodextrin (Cavitron), Unmicronized | 220 | |
| 5 | Magnesium trisilicate | 20 | |
| 6 | Hydroxyethyl Cellulose (Natrosol ® | 69 | |
| | 250M) | | |
| 7 | Simethicone Emulsion | 0.12 | |
| | Sub-Total, Unlubricated | 429 | |
| 8 | Magnesium Stearate (0.5% of | 2.145 | |
| | Unlubricated) | | |
| | Sub-Total, Core Weight | | 431.145 |
| 9 | Hydroxyethyl Cellulose (Natrosol ® | 5.17374 | |
| | 250L) | | |
| 10 | Hydroxypropyl methyl Cellulose | 4.31145 | |
| | (Opadry ® Clear) | | |
| 11 | Talc (Alphafil ®) | 0.64672 | |
| 12 | Polyethylene glycol 400 | 0.64672 | |
| | Sub-Coat Weight Gain | | 10.7786 |
| 13 | Opadry ® 03-B-53026 Orange Weight | | 10.7786 |
| | Gain | | |
| | Total, Coated Tablet Weight | | 452.702 |

### Example 6

| No. | Ingredient | mg/Unit | |
|---|---|---|---|
| 1 | Felodipine, BP, Micronized | 5 | |
| 2 | Hydroxypropyl Cellulose (Klucel ® LF) | 24.88 | |
| 3 | β-cyclodextrin (Cavitron), Micronized | 85 | |
| 4 | β-cyclodextrin (Cavitron), Unmicronized | 220 | |
| 5 | Magnesium trisilicate | 20 | |
| 6 | Hydroxyethyl Cellulose (Natrosol ® | 69 | |
| | 250M) | | |
| 7 | Simethicone Emulsion | 0.12 | |
| | Sub-Total, Unlubricated | 424 | |
| 8 | Magnesium Stearate (0.5% of | 2.12 | |
| | Unlubricated) | | |
| | Sub-Total, Core Weight | 426.12 | |
| 9 | Hydroxyethyl Cellulose (Natrosol ® | 5.11344 | |
| | 250L) | | |
| 10 | Hydroxypropyl methyl Cellulose | 4.2612 | |
| | (Opadry ® Clear) | | |
| 11 | Talc (Alphafil ®) | 0.63918 | |
| 12 | Polyethylene glycol 400 | 0.63918 | |
| | Sub-Coat Weight Gain | | 10.653 |
| 13 | Opadry ® 03-B-53026 Orange Weight | | 10.653 |
| | Gain | | |
| | Total, Coated Tablet Weight | | 447.426 |

### Example 7

| No. | Ingredient | mg/Unit | |
|---|---|---|---|
| 1 | Felodipine, BP, Micronized | 5 | |
| 2 | Hydroxypropyl Cellulose (Klucel ® LF) | 24.88 | |
| 3 | β-cyclodextrin (Cavitron), Micronized | 42.5 | |
| 4 | β-cyclodextrin (Cavitron), Unmicronized | 245 | |
| 5 | Magnesium trisilicate | 20 | |
| 6 | Hydroxyethyl Cellulose (Natrosol ® | 69 | |
| | 250M) | | |
| 7 | Simethicone Emulsion | 0.12 | |
| | Sub-Total, Unlubricated | 406.5 | |
| 8 | Magnesium Stearate (0.5% of | 2.0325 | |
| | Unlubricated) | | |
| | Sub-Total, Core Weight | | 408.533 |
| 9 | Hydroxyethyl Cellulose (Natrosol ® | 4.90239 | |
| | 250L) | | |
| 10 | Hydroxypropyl methyl Cellulose | 4.08533 | |
| | (Opadry ® Clear) | | |
| 11 | Talc (Alphafil ®) | 0.6128 | |
| 12 | Polyethylene glycol 400 | 0.6128 | |
| | Sub-Coat Weight Gain | | 10.2133 |
| 13 | Opadry ® 03-B-53026 Orange Weight | | 10.2133 |
| | Gain | | |
| | Total, Coated Tablet Weight | | 428.959 |

### Example 8

| No. | Ingredient | mg/Unit | |
|---|---|---|---|
| 1 | Felodipine, BP, Micronized | 5 | |
| 2 | Hydroxypropyl Cellulose (Klucel ® LF) | 24.88 | |
| 3 | β-cyclodextrin (Cavitron), Micronized | 42.5 | |
| 4 | β-cyclodextrin (Cavitron), Unmicronized | 232 | |
| 5 | Magnesium trisilicate | 20 | |
| 6 | Hydroxyethyl Cellulose (Natrosol ® | 81 | |
| | 250M) | | |
| 7 | Simethicone Emulsion | 0.12 | |
| | Sub-Total, Unlubricated | 405.5 | |
| 8 | Magnesium Stearate (0.5% of | 2.0275 | |
| | Unlubricated) | | |
| | Sub-Total, Core Weight | | 407.528 |
| 9 | Hydroxyethyl Cellulose (Natrosol ® | 4.89033 | |
| | 250L) | | |
| 10 | Hydroxypropyl methyl Cellulose | 4.07528 | |
| | (Opadry ® Clear) | | |
| 11 | Talc (Alphafil ®) | 0.61129 | |
| 12 | Polyethylene glycol 400 | 0.61129 | |
| | Sub-Coat Weight Gain | | 10.1882 |
| 13 | Opadry ® 03-B-53026 Orange Weight | | 10.1882 |
| | Gain | | |
| | Total, Coated Tablet Weight | | 427.904 |

### Example 9

Following the general procedure described herein the felodipine formulations in Table 9 were prepared and tableted. Formulation 9C used dibasic calcium phosphate dihydrate, in step 6, in place of magnesium trisilicate, used in formulation 9A. (As used herein, the term “FERT” refers to Felodipine Extended Release Tablets.)

TABLE 9

| Material | | | | |
|---|---|---|---|---|
| No. | Ingredient | | FERT-9C | FERT-9A |
| 1 | Felodipine | | 2.11 | 2.33 |
| 2 | β-cyclodextrin (β-CD) | | 44.47 | 70.93 |
| | (Cavitron ™ 82900) | | | |
| 3 | Hydroxyethyl cellulose | | 14.48 | 15.81 |
| | (Natrosol ® 250M) | | | |
| 4 | Hydroxypropyl cellulose | | 2.90 | 5.79 |
| | (Klucel ® LF) | | | |
| 5 | Dibasic calcium phosphate | | 34.26 | — |
| | dihydrate (Emcompress ®) | | | |
| 6 | Magnesium trisilicate, NF | | — | 4.65 |
| 7 | Simethicone Emulsion | | 0.04 | 0.03 |
| 8 | Magnesium stearate, NF | | 0.49 | 0.47 |
| 9 | Stearic acid, NF | | 0.97 | — |
| 10 | Colloidal silicone dioxide | | 0.29 | — |
| | TOTAL | | 100.0 | 100.0 |
| 11 | Film coating | | | |
| | I) | Opadry ® YS-1- | 2.5 | — |
| | | 10373A Purple | | |
| | ii) | Hydroxyethyl | — | 2.0 |
| | | cellulose | | |
| | | (Natrosol ® 250L) | | |
| | iii) | Talc (Alpha fill 500), | — | 0.2 |
| | | USP | | |
| | iv) | Polyethylene glycol | — | 0.2 |
| | | 400 | | |
| | v) | Opadry 03-B-53026 | — | 2.5 |
| | | Orange | | |

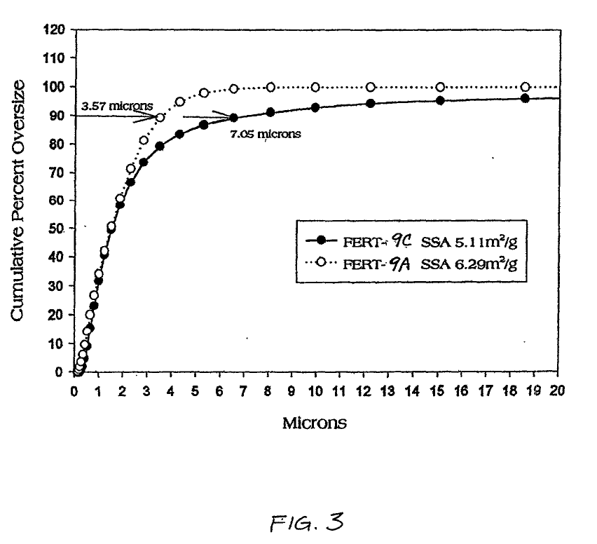

The formulation using magnesium trisilicate, 9A, was found to have d stability over the formulation using dibasic calcium phosphate dihydrate, 9C. The comparison of the particle size distributions for two drug dispersions after microfluidization are illustrated in FIG. 3, where SSA is the Specific Surface Area.

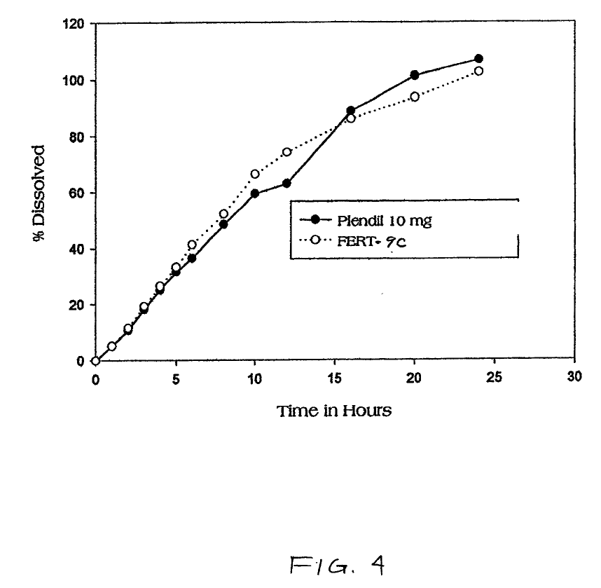

The dissolution profiles for the experimental dosage form 9C and a reference dosage form used in the pilot bioequivalence study are illustrated in FIG. 4. The particle size distribution of FERT 9A and FERT 9C are compared and reported in FIG. 3. Although release profiles are similar, the particle size was not optimum for achieving statistical bioequivalence between these dosage forms, FERT 9C and Plendil.

### Bulk Drug Stability

Bulk drug stability was evaluated in solution and solid state by exposing compositions in these states to accelerated conditions of stability (45° C. and 75% relative humidity). The stability of Felodipine blends with different excipients was studied at 60° C. and 45° C./75% RH, in a 1:5 ratio (felodipine: excipient). The excipients studied were β-cylodextrin (Cavitron®), Hydroxypropyl Cellulose (Klucel® LF), Dicalcium phosphate dihydrate (Emcompress®), Hydroxyethyl cellulose (Natrosol® 250M), Magnesium stearate (Hyqual®), Colloidal silicon dioxide (Aerosil® 200), Stearic acid (Hystren®), and total blend (blend of all excipients).

### Example 10

### Blend Stability in Suspension Form

The stability of felodipine suspensions, under conditions of 45° C. and 75% relative humidity, with various excipients, were studied in a ratio of 1:5 (drug: excipient). Results after 18 days and 30 days are illustrated in Table 10.

TABLE 10
Influence of β-Cyclodextrin on Suppressing the
Formation of “Impurity A” from Felodipine.

| | | Impurity | Unknown | % |
|---|---|---|---|---|
| Felodipine Blend | Conditions | A | Impurities | Potency |
| β-cyclodextrin | Initial | 0 | 0 | 98.42 |
| | 18 days, 45° C. | 0 | 0 | 98.25 |
| | 30 days, 45° C. | 0 | 0 | 98.39 |
| β-cyclodextrin + | Initial | 0 | 0 | 95.02 |
| Hydroxypropyl | 18 days, 45° C. | 0 | 0 | 94.79 |
| Cellulose | 30 days, 45° C. | 0 | 0 | 94.26 |
| β-cyclodextrin + | Initial | 0 | 0 | 91.28 |
| Hydroxypropyl | 18 days, 45° C. | 0 | 0 | 90.72 |
| Cellulose + | 30 days, 45° C. | 0 | 0 | 92.32 |
| Dicalcium Phosphate | | | | |
| Dihydrate | | | | |
| β-cyclodextrin + | Initial | 0 | 0 | 94.30 |
| Hydroxypropyl | 18 days, 45° C. | 0 | 0 | 94.59 |
| Cellulose + | 30 days, 45° C. | 0 | 0 | 93.61 |
| Hydroxyethyl | | | | |
| Cellulose | | | | |
| β-cyclodextrin + | Initial | 0 | 0 | 94.48 |
| Hydroxypropyl | 18 days, 45° C. | 0 | 0 | 94.56 |
| Cellulose + | 30 days, 45° C. | 0 | 0 | 95.06 |
| Magnesium Stearate | | | | |
| β-cyclodextrin + | Initial | 0 | 0 | 95.00 |
| Hydroxypropyl | 18 days, 45° C. | 0 | 0 | 94.45 |
| Cellulose + | 30 days, 45° C. | 0 | 0 | 95.27 |
| Colloidal Silicon | | | | |
| Dioxide | | | | |
| β-cyclodextrin + | Initial | 0 | 0.044, | 95.35 |
| Hydroxypropyl | | | 0.096 | |
| Cellulose + | 18 days, 45° C. | 0 | 0 | 94.85 |
| Stearic Acid | 30 days, 45° C. | 0 | 0 | 94.68 |
| Total blend | Initial | 0 | 0.014 | 93.96 |
| | 18 days, 45° C. | 0 | 0.392 | 93.19 |
| | 30 days, 45° C. | 0 | 0 | 93.21 |

The data in Table 10 illustrates that felodipine and β-cylodextrin form a stable non-covalently bonded composition.

### Example 11

A study was conducted to determine the pH of several combinations of excipients with and without felodipine to identify hydrogen ion (H⁺) sources. The heat treatment step involved heating the mixture at reflux for 15 minutes and allowing the mixture to stand over night.

TABLE 11
Dicalcium Phosphate Dihydrate Oxidation of Felodipine to Impurity “A”.

| | | Materials | Processing | | |
|---|---|---|---|---|---|
| No. | Materials Description | Composition | Conditions | pH | Δ pH |
| 1. | Purified water | Not applicable | Initial | 6.72 | +0.03 |
| | | Not applicable | Heat Treatment | 6.75 | |
| 2. | β-cyclodextrin | 60 g/100 ml | Initial | 6.11 | −0.89 |
| | | W/V | Heat Treatment | 5.22 | |
| 3. | Dibasic calcium | 46 g/100 ml | Initial | 7.39 | −3.32 |
| | phosphate (DCP) | W/V | Heat Treatment | 4.07 | |
| 4. | β-cyclodextrin + DCP | 46 g/100 ml | Initial | 7.23 | −3.41 |
| | | water W/V | Heat Treatment | 3.82 | |
| 5. | Felodipine + | 2.9 g + 60 g/ | Initial | 6.58 | −0.28 |
| | β-cyclodextrin | 100 ml water | Heat Treatment | 6.30 | |
| | | W/V | | | |
| 6. | Felodipine + | 2.9 g + 60 g + | Initial | 7.32 | −3.06 |
| | β-cyclodextrin + DCP | 46 g/100 ml | Heat Treatment | 4.26 | |
| | | W/V | | | |
| 7. | Felodipine + | 2.9 g + 60 g + | Initial | 10.20 | −0.24 |
| | β-cyclodextrin + Mg. | 5 g/100 ml | Heat Treatment | 9.96 | |
| | Stearate | W/V | | | |
| 8. | Felodipine + | 2.9 g + 60 g + | Initial | 9.98 | −1.37 |
| | β-cyclodextrin + Mg. | 11.5 g + 34.5 g/ | Heat Treatment | 8.61 | |
| | Stearate + DCP | 100 ml W/V | | | |

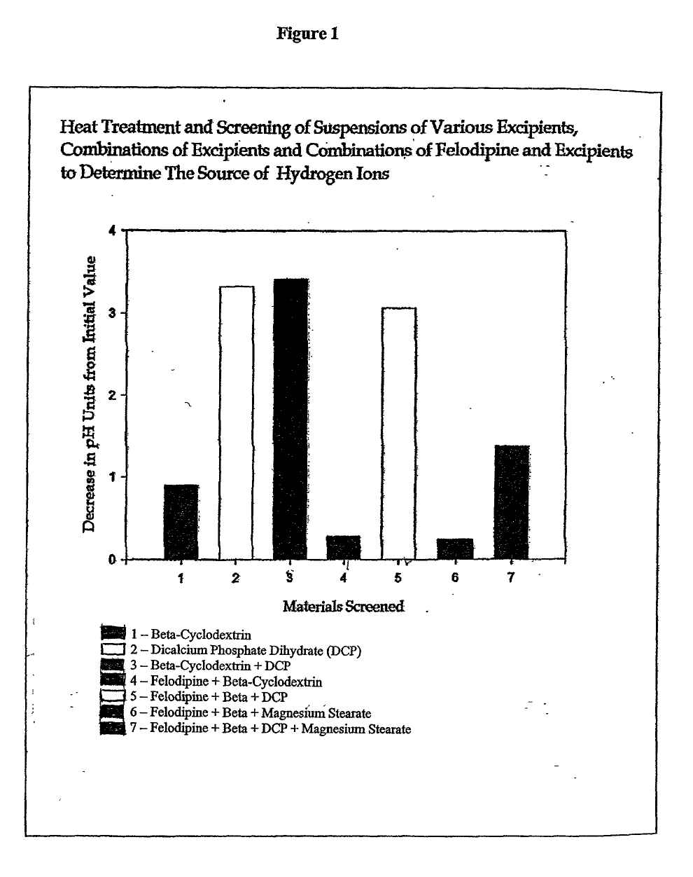

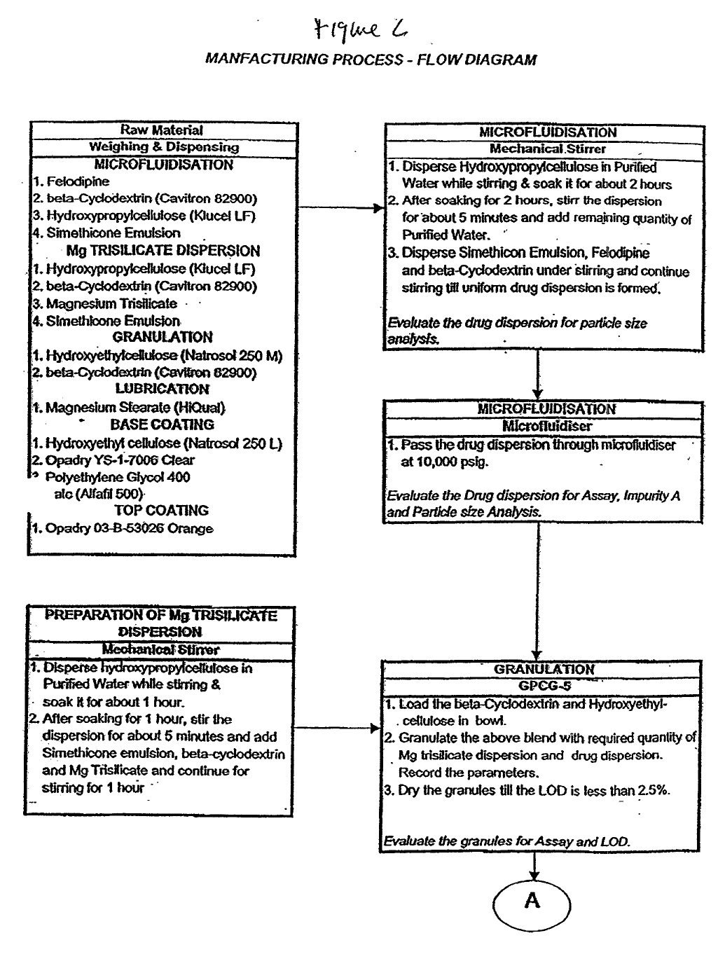

There was an unexpected finding discovered during experimentation when various excipients were heated at reflux for 15 minutes in the presence of felodipine and the suspension allowed to cool to room temperature. It was discovered that dicalcium phosphate dihydrate (DCP) individually, in combination with β-cyclodextrin, or in combination with felodipine and β-cyclodextrin (β-CD) degraded. The degradation products included a H⁺ion source which was indicated by a decrease the pH from the initial pH value of a specific material or a combination of materials. The order of pH decrease was β-cyclodextrin+DCP>DCP>Felodipine+β-cyclodextrin+DCP. When DCP was combined with β-cyclodextrin, the decrease in pH was the greatest. This is believed to be caused by the enhancement of the dissolution of DCP in the presence of β-cylodextrin. As felodipine is slightly alkaline in nature, the decrease in pH is less for a combination of Felodipine+β-cyclodextrin+DCP than a combination of β-cylodextrin+DCP alone. These results are graphically illustrated in FIG. 1.

### Example 12

A study was conducted to confirm, in actual product formulations, the results from Example 11, after heat treatment of suspensions of various excipients, combinations of excipients and combinations of felodipine and excipients was conducted. The basic formulation used was magnesium trisilicate 64.0 g, felodipine 180.0 g, β-cyclodextrin 1529.5 g (β-CD), hydroxypropyl cellulose 246.91 g and about 4380 g, remainder, purified water, for a final slurry weight of about 6400 g. The slurry had the 3.09 g simethicone added. The additional β-CD or DCP were added to this basic drug slurry. The heat treatment step involved heating the mixture at reflux for 15 minutes and allowing the mixture to stand over night.

TABLE 12
Heat Treatment and Screening of Raw Materials in Product Formulation.

| | Felodipine | | Processing | | |
|---|---|---|---|---|---|
| No. | Granulation Identity | Composition | Conditions | pH | Δ pH |
| 1. | Felodipine slurry | 5 g/100 ml | Initial | 8.40 | −2.99 |
| | Non Lubricated | | Heat Treatment | 5.41 | |
| | Granules plus | | | | |
| | β-CD + | | | | |
| | DCP (FERT-015) | | | | |
| 2. | Felodipine slurry | 5 g/100 ml | Initial | 8.69 | +0.17 |
| | Non Lubricated | | Heat Treatment | 8.86 | |
| | Granules plus β-CD | | | | |
| | alone. (FERT-016) | | | | |
| 3. | Felodipine slurry | 5 g/100 ml | Initial | 8.36 | |
| | Non Lubricated | | Heat Treatment | 5.10 | −3.26 |
| | Granules plus DCP | | | | |
| | alone. (FERT-017) | | | | |

The formulation FERT-015, was prepared using the basic felodipine slurry described above with β-cyclodextrin and DCP. The formulation FERT-016, was prepared using the basic felodipine slurry described above with β-cyclodextrin alone. The formulation FERT-017, was prepared using the basic felodipine slurry described above with DCP alone.

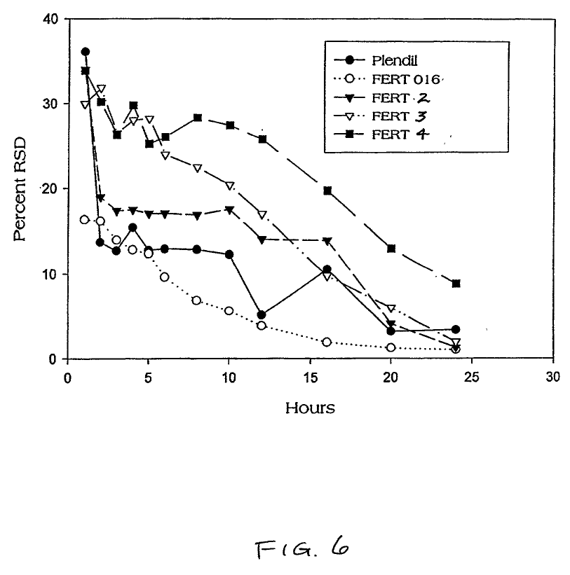

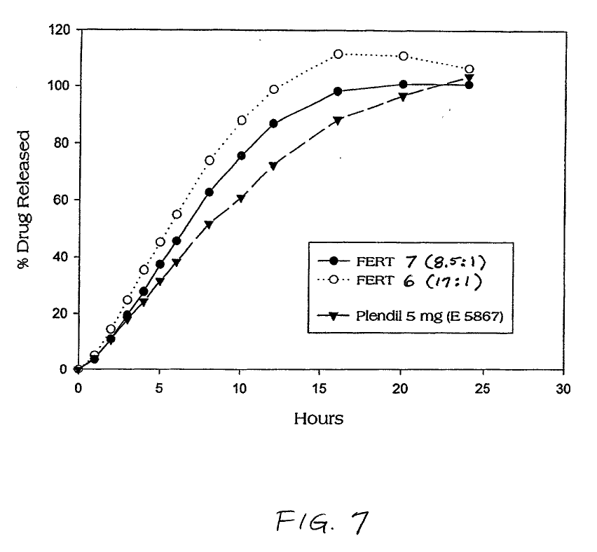

The dissolution profiles of formulations FERT 2, FERT 3, FERT 4, FERT 16 and Plendil were determined. The influence on the relative standard deviation (RSD) of drug release because of locating the unmicronized, powdered magnesium trisilicate in the “bowl charge” of the Fluid Bed Granulator Dryer vs. spraying it in a slurry form along with the drug slurry is illustrated in FIG. 6. Formulation FERT 16 contained 1% Magnesium Trisilicate in slurry-spray form. Formulation FERT 2 contained 8% Magnesium Trisilicate in bowl charge. Formulation FERT 3 contained 4.8% Magnesium Trisilicate in bowl charge. Formulation FERT 4 contained a 10.5% Magnesium Trisilicate in bowl charge

The results from this experiment confirmed that use of β-cylodextrin alone provided a more stable combination than the formulations that used DCP alone or DCP in combination with β-CD.

### Example 13

A study was conducted to determine the quantity of magnesium trisilicate required to neutralize the acid generated by degradation of DCP after heat treatment in a formulation. The mixtures, except the initial felodipine, B-CD and DCP formulation, were subjected to a heat treatment step that involved heating the mixture at reflux for 15 minutes and allowing the mixture to stand over night.

TABLE 13
Optimization of Magnesium Trisilicate in the Presence of DCP

| | | | Processing | |
|---|---|---|---|---|
| No. | Product/Description | Composition | Conditions | pH |
| 1. | Felodipine + β- | 2.9 g + 60 g + | Initial | 7.17 |
| | cyclodextrin + DCP | 46 g/100 ml | Heat Treatment | 3.88 |
| | Stock Slurry | W/V | | |
| 2. | Mg. Trisilicate | Stock Slurry | Initial | 3.88 |
| | a) 0.5% | +0.5 g | Heat Treatment | 4.62 |
| | b) 1.0% | +1.0 g | Heat Treatment | 5.00 |
| | c) 2.0% | +4.0 g | Heat Treatment | 7.23 |

The results of this experiment illustrate that the use of magnesium trisilicate significantly lowered the H⁺ ion availability and increased the pH of the formulations.

### Example 14

The formulation, FERT-16 containing β-cyclodextrin was prepared. A small amount, 1%, of magnesium trisilicate, in slurry form, as a water insoluble alkaline excipient was added. An accelerated study was performed by heating the formulation at 60° C. for 7 days and for 15 days. The impurities, Impurity A, dehydrogenated felodipine (pyridine) and the other impurities, Impurity B, the dimethyl ester of felodipine and Impurity C, the diethyl ester of felodipine were not significantly increased during the testing of the current formulation placed at accelerated conditions. The results of an are presented in Table 14.

TABLE 14
Product—Stability using β-Cyclodextrin

| | | | | | | |
|---|---|---|---|---|---|---|
| | Impurity A % | Impurity B % | Impurity C % | Unknown | Total | |
| Assay | (pyridine) | (dimethyl ester) | (diethyl ester) | Impurity | Impurities | LOD |

FERT-016 (β-cyclodextrin in Bowl charged with 1% Mg. Trisilicate in slurry)

| Initial | | 95.71 | 0.06 | 0.38 | 0.70 | Nil | 1.14 | 0.39 |
|---|---|---|---|---|---|---|---|---|
| 60° C. | 7 days | 96.1 | 0.10 | 0.42 | 0.67 | Nil | 1.19 | 1.58 |
| | 15 days | 95.5 | 0.07 | 0.38 | 0.71 | Nil | 1.16 | 0.77 |

The bowl charge comprised unmicronized β-cyclodextrin (Cavitron ™ 8900) and hydroxyethyl cellulose (Natrosol ® 250 M) in a 3:1 weight ratio.

### Example 15

### Degradation of Felodipine using Dicalcium Phosphate Dihydrate

A study was conducted to determine the effect of dicalcium phosphate dihydrate on felodipine degradation. The basic formulation used was magnesium trisilicate 64.0 g, felodipine 180.0 g, β-cylodextrin 1529.5 g (β-CD), hydroxypropyl cellulose 246.91 g and about 4380 g, remainder, purified water, for a final slurry weight of about 6400 g. The slurry had the 3.09 g simethicone added. Formulation FERT-015 (Table 15) was prepared by adding magnesium trisilicate to the FBGD bowl (bowl charge). Formulation FERT-017 (Table 15A) was prepared using only a minor amount, 1%, of magnesium trisilicate in the slurry. Formulation FERT-018 (Table 15B) was prepared using 3.5% of magnesium trisilicate in the FBGD bowl and no magnesium trisilicate in the slurry. The degradation of felodipine was monitored and the results are illustrated in Table 15, Table 15A and Table 15B:

TABLE 15

| | | | | | | |
|---|---|---|---|---|---|---|
| | Impurity | Impurity B % | Impurity C % | | | |
| | A % | (dimethyl | (diethyl | Unknown | Total | |
| Assay % | (pyridine) | ester) | ester) | Impurity % | Impurities % | LOD % |

FERT-015 (β-cyclodextrin + DCP in FBGD Bowl with 1% Mg. Trisilicate in slurry)

| Initial | | 102.75 | 010 | 0.43 | 0.75 | Nil | 1.28 | 0.76 |
|---|---|---|---|---|---|---|---|---|
| 60° C. | 7 days | 101.2 | 5.45 | 0.47 | 0.67 | 0.044, | 6.744 | 4.12 |
| | | | | | | 0.018, | | |
| | | | | | | 0.092 | | |
| | 15 | 99.4 | 6.34 | 0.41 | 0.76 | 0.018, 0.01, | 7.59 | 7.18 |
| | days | | | | | 0.031, 0.03 | | |

TABLE 15A

| | | | | | | |
|---|---|---|---|---|---|---|
| | Impurity | Impurity B % | Impurity C % | | | |
| | A% | (dimethyl | (diethyl | Unknown | Total | |
| Assay % | (pyridine) | ester) | ester) | Impurity % | Impurities % | LOD % |

FERT-017 (Dibasic calcium phosphate in FBGD Bowl with 1% Mg. Trisilicate in slurry)

| Initial | | 94.42 | 0.09 | 0.39 | 0.69 | Nil | 1.17 | 0.1 |
|---|---|---|---|---|---|---|---|---|
| 60° C. | 7 days | 94.00 | 1.42 | 0.42 | 0.65 | Nil | 2.49 | 5.43 |
| | 15 | — | — | — | — | — | — | — |
| | days | | | | | | | |

TABLE 15B
FERT-018 (β-cyclodextrin + DCP + 3.5% Mg. Trisilicate
in FBGD Bowl, No Mg. trisilicate in slurry)

| Initial | | 102.2 | 0.11 | 0.44 | 0.68 | Nil | 1.23 | 2.81 |
|---|---|---|---|---|---|---|---|---|
| 60° C. | 7 days | 93.5 | 6.44 | 0.39 | 0.69 | 0.022, | 7.582 | 5.97 |
| | | | | | | 0.04 | | |
| | 15 days | — | — | — | — | — | — | — |

### Example 16

Formulation FERT-019 contained β-cylodextrin along with a larger amount of magnesium trisilicate (13.3%) as the water insoluble alkaline excipient in the FBGD bowl (“bowl charge”). The results of accelerated study are presented below in Table 16.

TABLE 16

| | | | | | | |
|---|---|---|---|---|---|---|
| | | | | Unknown | Total | |
| Assay | Impurity A | Impurity B | Impurity C | Impurity | Impurities | LOD |

FERT-019 (β-cyclodextrin + 13.3% Mg. Trisilicate in Bowl charge, No Mg. Trisilicate in slurry)

| Initial | | 107.1 | 0.08 | 0.46 | 0.72 | Nil | 1.26 | 0.84 |
|---|---|---|---|---|---|---|---|---|
| 60° C. | 7 days | 106.5 | 0.12 | 0.43 | 0.72 | Nil | 1.27 | 0.87 |
| | 15 days | 105.39 | 0.13 | 0.85 | 0.74 | Nil | 1.72 | — |

The results presented in Examples 16 and 17 illustrate that the magnesium trisilicate can be useful in minimizing the conversion of felodipine to Impurity A in a tableted formulation.

### Example 17

### Bioequivalence Studies

Bioequivalence studies were conducted. In Bioequivalence Study 1, two dosage forms (one test and Plendil, reference) containing 10 mg of felodipine were administered as single doses in a crossover protocol to a group of 10 healthy male subjects on a fasted stomach. The test formulation 9C, was a 10 mg felodipine tablet prepared according to the present invention as described in example 9. The reference formulation was a 10 mg Plendil® Extended Release felodipine Tablet. The plasma concentrations of felodipine were compared with the plasma concentration after a single dose of Plendil® Extended Release Tablets. The particle diameter of the microfluidized dispersion was 7.05 micrometers (0.9) and 1.53 micrometers (0.5) for the Test Formulation in Pilot Bio 1. The parameters of bioequivalence are listed in terms of test to reference ratios and 90 percent confidence intervals 2-One Sided in Table 17 below:

TABLE 17
A two one-sided 90% Confidence Interval t-Test for the Ratio of means
(T/R) of Bioavailability Parameters

| | Ratio, Test/Reference | |
|---|---|---|
| PARAMETER | (T/R) | 90% Confidence Interval |
| AUC_{0-∞}¹ | 0.84 | 67-101 |
| Ln (AUC_{0-∞})² | 0.967 | 70-101 |
| Cₘₐₓ³ | 0.79 | 67-92 |
| Ln Cₘₐₓ⁴ | 0.79 | 66-85 |
| Tₘₐₓ⁵ | 1.28 | — |

The explanation of the parameters of bioequivalence is presented below:
¹AUC_{0-∞} indicates area under the plasma-time curve and is computed using either trapezoidal rule or by defining the curve as a mathematical function (y as a function of x) and then integrating the function. (Nanogram-hours/ml)
²Ln (AUC_{0-∞}) is the natural logarithm of the AUC_{0-∞} value. (Nanogram-hours/ml)
³Cₘₐₓ indicates the peak value for drug concentration C in plasma. (Nanograms/ml)
⁴Ln Cₘₐₓ is the natural logarithm of the Cₘₐₓ value. (Nanogram/ml)
⁵Tₘₐₓ indicates the time on the x-axis where the peak value for drug concentration on the y-axis occurs.

Bioequivalence Study 2 was conducted with 15 fasted subjects. The test formulation 9A, was a 10 mg felodipine tablet prepared according to the present invention as described in example 9. The reference formulation was a 10 mg Plendil® Extended Release felodipine Tablet. The results for the parameters of bioequivalence are presented in Table 18:

TABLE 18
A two one-sided 90% Confidence Interval t-Test for the Ratio of means
(T/R) of Bioavailability Parameters

| PARAMETER | Ratio, (T/R) | 90% Confidence Interval |
|---|---|---|
| AUC_{0-∞} | 1.02 | 90.56-112.79 |
| Ln (AUC_{0-∞}) | 0.99 | 87.07-106.01 |
| Cₘₐₓ | 1.01 | 90.19-111.95 |
| Ln Cₘₐₓ | 1.01 | 90.96-112.15 |

The results illustrate that the erosion rate-controlled drug delivery system of the present invention is bioequivalent to the commercially available Plendil® tablet which is prepared by using felodipine and Cremophor® RH 40, and then delivering the solubilized drug from a gelling matrix comprised of low viscosity hydroxypropyl methyl cellulose (traded as Methocel® K50) along with excipients.

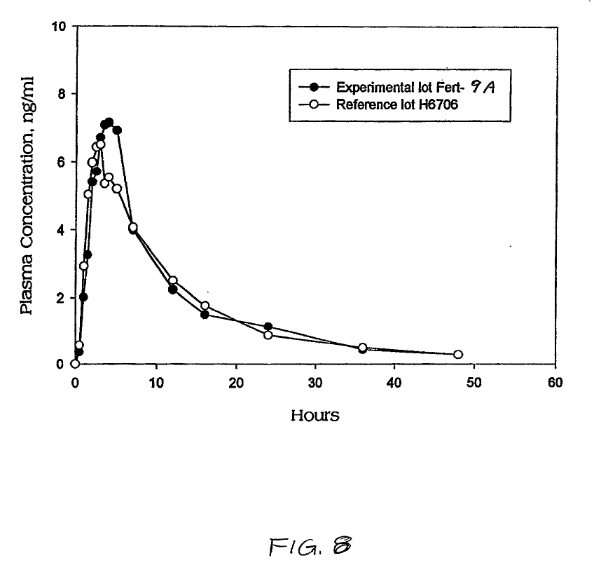

The release of felodipine is illustrated in FIG. 8 where the results of the bioequivalence comparison of felodipine tablets, prepared in Example 9A is compared with the reference Plendil tablets.

All publications, patents, and patent documents are incorporated by reference herein, as though individually incorporated by reference. In the case of any inconsistencies, the present disclosure, including any definitions therein will prevail. The invention has been described with reference to various specific and preferred embodiments and techniques. However, it should be understood that many variations and modifications may be made while remaining within the spirit and scope of the invention.

What is claimed is:

1. A method for preparing a unit dosage form of felodipine comprising forming a drug containing core by compressing a composition comprising granulated microparticles of felodipine and cyclodextrin, having a diameter of from about 0.5 microns to about 9 microns, wherein the felodipine particles are non-covalently bonded to the cyclodextrin; and
a carrier comprising cyclodextrin particles, a water-insoluble alkaline component and a swellable polymer.

2. The method of claim 1, wherein the microparticles comprising felodipine and cyclodextrin are formed by microfluidization.

3. The method of claim 1, wherein the cyclodextrin particles and the water-insoluble alkaline component are blended and microfluidized before addition of the swellable polymer.

4. The method of claim 1, wherein the microp articles comprise a felodipine-β-cyclodextrin component having a specific surface area (SSA) of from about 3 m²/g to about 7 m²/g.

5. The method of claim 4, wherein the microp articles comprise a felodipine-β-cyclodextrin component having a specific surface area (SSA) of from about 4 m²/g to about 8 m²/g.

6. The method of claim 4, wherein the microparticles comprise a felodipine-β-cyclodextrin component having a specific surface area (SSA) of from about 5 m²/g to about 7.5 m²/g.

7. The method of claim 1 wherein the water-insoluble alkaline component has a specific surface area (SSA) of from about 1 m²/g to about 3 m²/g.

8. The method of claim 1, wherein the cyclodextrin is selected from the group consisting of α-cyclodextrin, a-cyclodextrin, dimethyl β-cyclodextrin and hydroxypropyl a-cyclodextrin.

9. The method of claim 8, wherein the cyclodextrin comprises a-cyclodextrin.

10. The method of claim 1, wherein the solid dosage form comprises from about 1 mg to about 15 mg of felodipine.

11. The method of claim 10, wherein the solid dosage form comprises from about 2.5 mg to about 10 mg of felodipine.

12. The method of claim 1, wherein the solid dosage form is a tablet.

13. The method of claim 12, wherein the tablet is coated with a non-enteric coating.

14. The method of claim 13, wherein the coating comprises a resilient membrane.

15. The method of claim 14, wherein the coating comprises a film forming polymer selected from the group consisting of hydroxypropyl methyl cellulose and hydroxyethyl cellulose.

16. The method of claim 15, wherein the coating comprises a mixture of hydroxypropyl methyl cellulose and hydroxyethyl cellulose.

17. The method of claim 1, wherein the a-cyclodextrin comprises from about 40 to about 80 weight percent of the composition based on the total weight of the composition.

18. The method of claim 17, wherein the a-cyclodextrin comprises from about 50 to about 75 weight percent of the composition based on the total weight of the composition.

19. The method of claim 18, wherein the a-cyclodextrin comprises is about 60 to about 70 weight percent of the composition based on the total weight of the composition.

20. The method of claim 1, wherein the microparticles of felodipine and cyclodextrin further comprise a binder.

21. The method of claim 20, wherein the binder is selected from the group consisting of a hydroxyalkyl cellulose, polyvinylpyrrolidone, gelatin, and acacia.

22. The method of claim 21, wherein the binder is hydroxyalkyl cellulose.

23. The method of claim 22, wherein the binder comprises hydroxypropyl cellulose.

24. The method of claim 1, wherein the swellable polymer is an alginate, carrageenan, pectin, guar gum, xanthan gum, modified starch or hydroxyalkyl cellulose.

25. The method of claim 24, wherein the swellable polymer is hydroxyalkylcellulose.

26. The method of claim 25, wherein the hydroxyalkylcellulose is hydroxypropylmethyl cellulose, hydroxypropyl cellulose, sodium carboxymethyl cellulose or hydroxyethyl cellulose.

27. The method of claim 26, wherein the hydroxyalkylcellulose is hydroxyethyl cellulose

28. The method of claim 1, wherein the alkaline agent is selected from the group consisting of oxides, hydroxide salts, carbonate salts, and trisilicate salts of basic cations.

29. The method of claim 27, wherein the basic cation is selected from the group consisting of magnesium, calcium, and aluminum.

30. The method of claim 29, wherein the alkaline agent is selected from the group consisting magnesium oxide, magnesium trisilicate, aluminum hydroxide, magnesium hydroxide and magnesium aluminum silicate.

31. The method of claim 30, wherein the alkaline agent is magnesium trisilicate.

32. The method of claim 31, wherein amount of magnesium trisilicate is from about 0.5 to about 15 percent of the weight of the composition.

33. The method of claim 32, wherein amount of alkaline agent is from about 2 to about 10 percent.

34. The method of claim 33, wherein amount of alkaline agent is from about 3 to about 8 percent.

35. The method of claim 1, wherein the composition is substantially free of dicalcium phosphate.
